(19) United States
(12) Patent Application Publication
(10) Pub. No.: US 2022/0330543 A1
(43) Pub. Date: Oct. 20, 2022

(54) METHODS OF BULK DROPLET VITRIFICATION

(71) Applicant: The General Hospital Corporation, Boston, MA (US)

(72) Inventors: Mehmet Toner, Charlestown, MA (US); Lindong Weng, Arlington, MA (US); Reinier Johan De Vries, Boston, MA (US); Shannon N. Tessier, Framingham, MA (US); Mustafa Uygun, Newton, MA (US)

(21) Appl. No.: 17/632,322

(22) PCT Filed: Dec. 2, 2019
(86) PCT No.: PCT/US2019/064026

§ 371 Date: Feb. 2, 2022

Related U.S. Application Data
(60) Provisional application No. 62/882,073, filed on Aug. 2, 2019.

(51) Int. Cl. A01N 1/02

(52) U.S. Cl. CPC A01N 1/0284; A01N 1/0221; A01N 1/0252

(57) ABSTRACT

The present disclosure provides methods for bulk droplet vitrification of cells, compositions including the vitrified droplets, and systems for performing the methods for bulk droplet vitrification cells.

### CLAIM OF PRIORITY

This application claims the benefit of U.S. Provisional Patent Application Ser. No. 62/882,073, filed on Aug. 2, 2019. The entire contents of the foregoing application are hereby incorporated by reference.

### FEDERALLY SPONSORED RESEARCH OR DEVELOPMENT

This invention was made with Government support under Grant Nos. R01DK096075, R01DK107875, and R01DK114506 awarded by the National Institutes of Health. The Government has certain rights in the invention.

### TECHNICAL FIELD

Provided herein are methods and compositions for cryopreserving cells.

### BACKGROUND

End-stage liver disease claims over 30,000 lives in the U.S. every year. Many patients become too ill to tolerate liver transplantation, and even if transplantation were a viable solution, there is a severe shortage of donor organs whereby only 28% of the wait-listed patients receive transplants. As bio-artificial liver assist devices (BALs) enter clinical trials, hepatocyte transplantation continues as an alternative treatment to liver failure, and whole-organ tissue engineering emerges as a vertical-advancement in the field, where identifying suitable sources of hepatocytes is becoming an immediate issue (9). For example, BAL devices require about 10¹⁰ hepatocytes and up to 10⁹ hepatocytes are required per transplantation infusion. These cells must be highly viable to avoid potential harm to an already ill patient (4, 10).

However, a major bottleneck in translating these ideally “off-the-shelf” cell-based treatment technologies to the clinic is the absence of a method to preserve them long enough for preparation and transportation to the patient site without considerable loss of hepatocyte viability and function (11).

The classic method for cryopreservation is slow freezing after pre-incubation of one or more cryoprotectant agents (CPAs) (12, 13). However, CPA toxicity coupled with mechanical and osmotic stress of extracellular ice crystallization and recrystallization remain fundamental problems in cryopreservation. Significant loss of viability and metabolic activity is still a major issue (4, 10, 15).

Vitrification can avoid ice-mediated injury via a direct transition from the aqueous to the glass phase (12). However, extremely fast cooling rates are required to reach the glass transition temperature while avoiding ice formation at higher subzero temperatures (16, 17). By addition of CPAs, the required critical cooling rate can be reduced, and the glass transition temperature increased (16). Nonetheless, the required CPA concentrations are high, typically over 40% (v/v) (18). To reach such high concentrations, stepwise CPA introduction is used to reduce osmotic injury during CPA incubation (12). However, classical vitrification is a cumbersome and time-consuming technique, which is practically constrained to simultaneous processing of a few samples. Moreover, it considerably extends the exposure time of cells to high toxic CPA concentrations. Altogether, CPA toxicity is one of the key limitations of vitrification (20, 21).

Although the critical cooling rate of aqueous solutions can be reduced by the addition of CPAs, extremely fast cooling rates of several hundred degrees per minute are nonetheless required for successful vitrification (17). To obtain these high cooling rates, the samples must have small volumes (microliters) and large surface areas and are therefore intrinsically unsuitable for scale-up efforts to large volumes.

### SUMMARY

The present disclosure features methods for bulk droplet vitrification of cells, e.g., hepatocytes, such that the cells are pre-incubated with low concentrations cryoprotective agents (“CPA”) for short time intervals resulting in increased viability post-preservation as compared to cryopreservation by slow freezing. Prior to vitrification of droplets of cells, the cells are incubated in a first, low concentration CPA solution. After this initial pre-incubation the cells are briefly (e.g., less than one minute) exposed to a second, high CPA concentration (e.g., at least 30%) solution and directly bulk vitrified in liquid nitrogen to limit the exposure time to high concentrations of CPA throughout the entire process.

In particular, in one aspect, the disclosure features methods of bulk droplet vitrification of cells, e.g., hepatocytes, the methods including (a) incubating a plurality of cells in a first cryoprotective solution including one or more cryoprotectant agents (CPAs) at a concentration of about 20% or less (v/v); (b) mixing the plurality of cells in the first cryoprotective solution with a second cryoprotective solution including one or more CPAs at a concentration of greater than about 30% (v/v) and generating a plurality of droplets of the resulting mixture within less than one minute, e.g., less than 50, 40, 30, 20, 15, 10, or 5 seconds, from the start of mixing, wherein at least some of the droplets contain one or more of the cells; and (c) rapidly cooling the plurality of droplets in a cooling liquid at a cooling rate of faster than 0.1° C./second, e.g., faster than 30, 50, 75, 100, 200, 300, 400, 500, 600, 700, or 800° C./minute, for a time sufficient to bulk vitrify the droplets including cells.

In some embodiments of the methods, the concentration of the CPA of the first solution is less than about 15% (v/v), less than about 10% (v/v), less than about 5% (v/v), or less than about 1% (v/v). For example, the first solution can include between 5% (v/v) and 10% (v/v) dimethyl sulfoxide (DMSO) and between 5% (v/v) and 10% (v/v) ethylene glycol, e.g., the first solution includes about 7.5% (v/v) DMSO and about 7.5% (v/v) ethylene glycol. In some implementations, the first solution further includes University of Wisconsin solution (UW solution) and/or bovine serum albumin (BSA).

In certain embodiments, the second solution includes greater than 20% (v/v) DMSO, greater than 20% (v/v) ethylene glycol, and greater than 500 mM sucrose. For example, the second solution can include about 35% (v/v) DMSO, about 35% (v/v) ethylene glycol, and about 800 mM sucrose. In some implementations, the second solution further includes UW solution and/or BSA.

In certain embodiments, droplets in the plurality of droplets have an average diameter of between about 0.5 mm and about 10 mm, optionally between about 1 mm and about 6 mm or between 2 mm and 4 mm. In some implementations, the droplets include between 10-30% (v/v) DMSO, about 10-30% (v/v) ethylene glycol, and about 200-600 mM sucrose. For example, the droplets can include about 20% (v/v) DMSO, about 20% (v/v) ethylene glycol, and about 400 mM sucrose.

In some embodiments, the mixing and droplet formation occurs in less than 5 seconds, e.g., in less than 2 seconds. In some implementations, the cooling rate is between about 900° C./minute and 1400° C./minute. In some embodiments, the droplets are cooled to a temperature of about −180° C. to about −210° C. In certain embodiments, the cooling liquid includes liquid nitrogen.

In the new methods, the vitrified cells can have greater than 75% cell viability after rewarming, as measured by assessing membrane integrity of the cells. The methods can be carried out such that the vitrified droplets are generated continuously from the mixture of the first solution and the second solution at a volumetric flow rate of least 4 ml/minute of the mixture being used to form the vitrified droplets per minute.

In another aspect, the disclosure features droplet generation and vitrification systems that include a first vessel for containing a first solution; a second vessel for containing a second solution; a mixing and droplet generation chamber including an inlet connected to both the first vessel and the second vessel and further including an outlet, wherein the mixing and droplet generation chamber is configured to receive and mix the first solution and the second solution and to expel through the outlet droplets of the mixture of the first solution and the second solution; a cooling liquid reservoir arranged to receive droplets expelled from the mixing and droplet generating chamber outlet; and a pressure source arranged to flow the first and second solutions from the first and second vessels into and through the mixing and droplet generation chamber, and controlled to flow the mixture from the inlet to the outlet of the mixing and droplet generation chamber within less than 10 seconds.

In some embodiments of the systems, the first and second vessels and the mixing and droplet generation chamber outlet are arranged a distance above the cooling container, such that the droplets fall from the outlet of the mixing and droplet generation chamber into the cooling liquid reservoir.

In certain implementations, the outlet of the mixing and droplet generation chamber is sized, and the pressure source is controlled, to generate droplets with an average diameter of between about 0.5 mm and about 10 mm, e.g., between about 2 mm and 4 mm. In some implementations, the pressure source is controlled to flow the mixture from the inlet to the outlet of the mixing and droplet generation chamber within less than 2 seconds.

In some embodiments of the systems, the cooling liquid reservoir contains a cooling liquid, e.g., one or more of liquid nitrogen, liquid isopentane, and liquid propane, for example cooled to a temperature of between about −180° C. and about −210° C.

In another aspect, the disclosure features compositions that include a plurality of vitrified droplets made by the any of the methods described herein. In these compositions, the droplets can have an average diameter of between about 0.5 mm and about 10 mm, optionally between about 2 mm and about 4 mm. In certain embodiments, the droplets include cells, such as hepatocytes. In some embodiments, the droplets in the compositions include between 10-30% (v/v) DMSO, about 10-30% (v/v) ethylene glycol, and about 200-600 mM sucrose. For example, the droplets can include about 20% (v/v) DMSO, about 20% (v/v) ethylene glycol, and about 400 mM sucrose.

In any of these compositions, the vitrified cells can have greater than 75% cell viability, as measured by assessing membrane integrity of the cells.

The methods described herein provide numerous benefits and advantages. For example, an important benefit it that vitrification is an ice-free cryopreservation method that therefore completely avoids freezing injury. Another advantage of the new methods is that—unlike most other vitrification approaches—the exposure of the cells to CPAs in high concentrations is limited thereby increasing post-preservation viability. Also, this method enables processing of large cell volumes. Bulk droplet vitrification is a simple and cost-effective preservation method that can be used to preserve a variety of different cell types, e.g., mammalian, avian, fish, reptile, or amphibian cell types.

As described herein, the term “cryoprotectant agent” (CPA) refers to one or more chemicals, proteins, and/or polymers that can provide protection to cells from adverse effects of subzero temperatures, e.g., by reducing ice crystal formation within and outside the cells. In some embodiments, a CPA can be dimethyl sulfoxide (DMSO), polyvinylpyrrolidone (PVP), glycerol, ethylene glycol (EG), propylene glycol (PG), propanediol (PROH), methyl pentanediol, polyethylene glycol (PEG), hydroxyethyl starch (HES), sucrose, sucralose, mannitol, maltose, glucose, 3-O-Methyl-D-glucose, trehalose, dextrose, or any combination thereof.

As described herein, University of Wisconsin solution (UW solution) includes 50 g/L pentafraction, 35.83 g/L lactobionic acid (as Lactone), 3.4 g/L potassium phosphate monobasic, 1.23 g/L magnesium sulfate heptahydrate, 17.83 g/L raffinose pentahydrate, 1.34 g/L adenosine, 0.136 g/L allopurinol, 0.922 g/L total glutathione, 5.61 g/L potassium hydroxide, and adjusted to pH 7.4.

As described herein, the “first cryoprotective solution” includes one or more CPAs at a concentration of 20% or less than 20%. In some embodiments, the first solution includes one or more CPAs at a concentration of 19.5% (v/v), 19% (v/v), 18.5% (v/v), 18% (v/v), 17.5% (v/v), 17% (v/v), 16.5% (v/v), 16% (v/v), 15.5% (v/v), 15% (v/v), 14.5% (v/v), 14% (v/v), 13.5% (v/v), 13% (v/v), 12.5% (v/v), 12% (v/v), 11.5% (v/v), 11% (v/v), 10.5% (v/v), 10% (v/v), 9.5% (v/v), 9% (v/v), 8.5% (v/v), 8% (v/v), 7.5% (v/v), 7% (v/v), 6.5% (v/v), 6% (v/v), 5.5% (v/v), 5% (v/v), 4.5% (v/v), 4% (v/v), 3.5% (v/v), 3% (v/v), 2.5% (v/v), 2% (v/v), 1.5% (v/v), 1% (v/v), and 0.5% (v/v).

As described herein, the “second cryoprotective solution” includes one or more CPAs at a concentration of at least 30%. In some embodiments, the second solution includes one or more CPA at a concentration of 31% (v/v), 32% (v/v), 33% (v/v), 34% (v/v), 35% (v/v), 36% (v/v), 37% (v/v), 38% (v/v), 39% (v/v), 40% (v/v), 41% (v/v), 42% (v/v), 43% (v/v), 44% (v/v), 45% (v/v), 46% (v/v), 47% (v/v), 48% (v/v), 49% (v/v), 50% (v/v), 51% (v/v), 52% (v/v), 53% (v/v), 54% (v/v), 55% (v/v), 56% (v/v), 57% (v/v), 58% (v/v), 59% (v/v), 60% (v/v), 61% (v/v), 62% (v/v), 63% (v/v), 64% (v/v), 65% (v/v), 66% (v/v), 67% (v/v), 68% (v/v), 69% (v/v), 70% (v/v), 71% (v/v), 72% (v/v), 73% (v/v), 74% (v/v), 75% (v/v), 76% (v/v), 77% (v/v), 78% (v/v), 79% (v/v), 80% (v/v), 81% (v/v), 82% (v/v), 83% (v/v), 84% (v/v), 85% (v/v), 86% (v/v), 87% (v/v), 88% (v/v), 89% (v/v), 90% (v/v), and 100% (v/v).

As used herein, the terms “bulk droplet vitrification,” bulk vitrification,” and “vitrification” mean the rapid cooling of droplets containing cells in a manner that avoids ice crystal formation within the droplets and within the cells, but includes the formation of minute amounts of ice crystals within the droplets and/or cells that are not deleterious to cell viability.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic representation of the bulk droplet vitrification method.

FIG. 2 is a representation of a bulk droplet vitrification experimental setup. Insert a: detail of the mixing and droplet generation chamber. Insert b: droplet collection device, which is placed in the liquid nitrogen filled dewar.

FIG. 3A is a graph of droplet diameter measurements.

FIG. 3B is a graph of droplet temperature over time after exposure to liquid nitrogen at t=0 seconds.

FIG. 3C is a graph of relative volume change of hepatocytes during CPA incubation with exposure to 7.5% (v/v) dimethyl sulfoxide (DMSO) from 0-3 minutes, 15% (v/v) DMSO from 3-6 minutes and 40% (v/v) DMSO with 400 mM sucrose from 6-7 minutes.

FIG. 3D is a graph of intracellular DMSO concentration over time during CPA incubation.

FIG. 4A is a bar graph of direct post-preservation viability of cryopreserved (blue) and bulk droplet vitrified (green) hepatocytes in suspension. Stars: p<0.05 Error bars: SD.

FIG. 4B is a bar graph of post-preservation yield percentage of cryopreserved (blue) and bulk droplet vitrified (green) hepatocytes in suspension. Stars: p<0.05 Error bars: SD.

FIG. 5A are representative images of Hoechst (all hepatocytes)/propidium iodide (PI) (dead hepatocytes) staining of cryopreserved (blue) and bulk droplet vitrified (green) hepatocytes after 24 hours monolayer culture. Scale bars: 400 μm. Stars: p<0.05 Error bars: SD.

FIG. 5B is a bar graph of total number of attached hepatocytes per field of view. Stars: p<0.05 Error bars: SD.

FIG. 5C is a bar graph of viability of attached hepatocytes. Stars: p<0.05 Error bars: SD.

FIG. 6A are representative microscopy images of fresh, cryopreserved and bulk droplet vitrified hepatocytes. Scale bars: 400 μm.

FIG. 6B is a bar graph of metabolic reduction activity of Presto Blue in collagen sandwich culture. Stars: p<0.05 Error bars: SD.

FIG. 7A is a bar graph of urea production in hepatocytes during long-term collagen sandwich cultures of fresh (grey), cryopreserved (blue) and bulk droplet vitrified (green) hepatocytes. Letters: p<0.05; a: fresh vs cryopreserved; b: fresh vs bulk droplet vitrified; c: cryopreserved vs bulk droplet vitrified. Error bars: SD.

FIG. 7B is a bar graph of albumin synthesis in hepatocytes during long-term collagen sandwich cultures of fresh (grey), cryopreserved (blue) and bulk droplet vitrified (green) hepatocytes. Letters: p<0.05; a: fresh vs cryopreserved; b: fresh vs bulk droplet vitrified; c: cryopreserved vs bulk droplet vitrified. Error bars: SD.

### DETAILED DESCRIPTION

Loss of hepatocyte viability and metabolic function after cryopreservation is still a major issue. The present disclosure provides novel bulk droplet (e.g., 2 to 6 mm diameter) vitrification methods that allow high throughput volumetric flow rates (e.g., at least 4 ml/min, e.g., 5 ml/min, or 6 ml/min, or faster, while using a low pre-incubated CPA concentration (e.g., 15%-20% v/v) and cooling at a rapid rate (e.g., faster than 0.1° C./second, e.g., faster than 6, 7, 8, 9, 10, 15, 20, 25, 30, 40, 50, 60, 70, 75, 100, 200, 300, 400, 500, 600, 700, 800, 900, 1000, 1100, 1200, 1300, 1400, or 1500° C./minute, e.g., wherein the cooling rate is between about 900° C./minute and 1400° C./minute) to minimize toxicity and loss of cell viability and function (17). In general, the cooling rate is directly related to the used CPA type and concentration. For example, the cooling rate for 30% DMSO is ˜100° C./second, while for 60% DMSO the cooling rate is “only” 0.1C/second.

The methods use rapid (e.g., less than one minute, e.g., less than 50, 40, 30, 20, 10, or 5 seconds) osmotic dehydration to concentrate a low pre-incubated intracellular CPA concentration ahead of vitrification, without the need of fully equilibrating toxic CPA concentrations.

Classical vitrification exposes cells to permeable CPAs, which leads to fast (seconds) dehydration followed by slower (minutes) rehydration when CPAs, together with water, diffuse into the cells (19). This effect is caused by the much faster diffusion rate of water over cell membranes as compared to CPAs. Multiple steps of CPA incubation leads to increased exposure to toxic CPA, slower production rates, and decreased post-preservation viability.

Compared to cryopreserved hepatocytes, bulk droplet vitrified hepatocytes prepared as described herein have a significantly higher viability, better morphology, and significantly higher metabolic activity than cryopreserved hepatocytes directly after preservation and after one day in culture. Simulations and cooling rate measurements confirmed an adequate concentration of the intracellular CPA concentration (e.g., up to 8.53 M) after dehydration in combination with high cooling rates (e.g., 960 to 1320° C./min) for successful vitrification.

### General Methodology

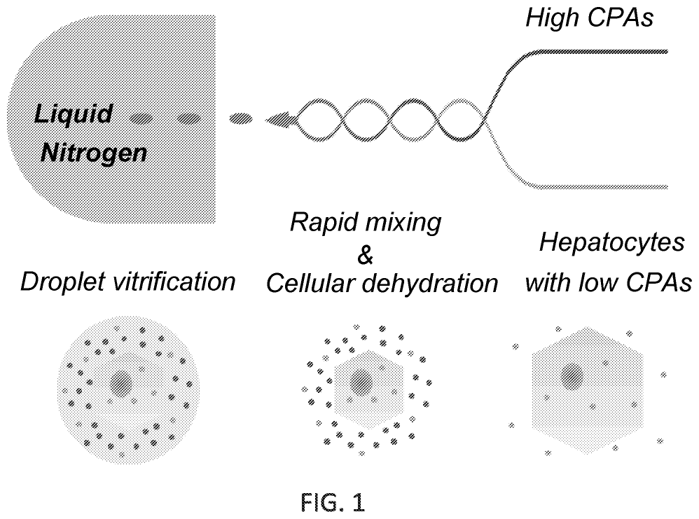

Here we present a novel bulk droplet vitrification technique, which allows vitrification of very large volumes of cells and droplets while limiting the exposure of the cells to CPAs. Using a brief osmotic dehydration seconds ahead of vitrification, we increased a low pre-incubated intracellular CPA concentration without the need of fully equilibrating high toxic CPAs concentrations. This is accomplished by loading low CPA pre-incubated cells (i.e., hepatocytes, red blood cells (RBs), white blood cells (WBCs), CAR-T cells, mammalian cells, and any cell in suspension), e.g., in a first low CPA solution, into high CPA concentration droplets, instantaneously followed by exposure to liquid nitrogen (FIG. 1). We confirmed that a dehydration for 1.25 seconds sufficiently increases the pre-incubated intracellular CPA concentration, which in conjunction with the measured cooling rates of our large size droplets enables vitrification (17). As such, we leveraged the rapid diffusion rate of water over cell membranes in comparison to CPAs.

The first solution can be UW solution or other commercially available flushing and cold storage preservation solutions for cells and organs, such as Celsior® (Mannitol 60.0 mmol/L, Lactobionic Acid 80.0 mmol/L, Glutamic Acid 20.0 mmol/L, Histidine 30.0 mmol/L, Calcium Chloride 0.25 mmol/L, Potassium Chloride 15.0 mmol/L, Magnesium Chloride 13.0 mmol/L, Sodium Hydroxide 100.0 mmol/L, Reduced Glutathione 3.0 mmol/L, Water for Injection (WFI) Up to 1 liter), Perfadex® (physiological salt solution, dextran 40, THAM buffer (0.3 M solution of tromethamine)), Somali® (Calcium chloride, Potassium chloride,Magnesium chloride (hexahydrate), Magnesium sulfate (heptahydrate) Sodium chloride, Sodium bicarbonate, Sodium phosphate (dibasic; heptahydrate), d-Glucose, Glutathione (reduced), Ascorbic acid, 1-Arginine, 1-Citrulline malate, Creatine orotate, Creatine monohydrate, 1-Carnosine, 1-Carnitine, Dichloroacetate, Insulin 10 mg/mL, mL/L), Histidine-tryptophan-ketoglutarate, Unisol™ (Sodium, Potassium, Calcium, Magnesium, Chloride, Bicarbonate Buffer (Carbonic Acid and Bicarbonate), HEPES Buffer (4-(2-hydroxyethyl)-1-piperazineethanesulfonic acid), Lactobionate, Sucrose, Mannitol, Glucose, Gluconate, Dextran 40, Adenosine, Glutathione, at pH 7.6), or Hypothermosol® (Sucrose, Dextran, 4-O-β-D-galactopyranosyl-D-gluconic acid, Sodium Hydroxide, Potassium Hydroxide).

Moreover, the high extracellular CPA concentration allows the vitrification of magnitudes larger sized droplets (e.g., 15-65 μl) than other droplet vitrification approaches, enabling vitrification of bulk volumes. For example, compared to micro-droplet vitrification, we were able to use over ten thousand times larger droplets resulting in much higher volume processing rates. The present disclosure provides a high throughput (e.g., at least 4 ml/min), low toxicity method for vitrification which may result in a protocol viable for use in clinical hepatocyte therapy studies.

In some embodiments, the cells are pre-incubated with low concentration CPA in a mixing chamber.

Contamination is a potential issue when using an open method such as droplet vitrification. To prevent contamination, we used our system in a sterile laminar flow cell culture hood. Although the liquid nitrogen used was not sterile, we did not encounter any contamination issues that would been revealed during long-term cultures. If required, liquid nitrogen can be sterilized by radiation or filtering (41).

Although high cooling rates are required for vitrification, it is well known that it is especially difficult to obtain high enough rewarming rates for successful rewarming (13, 20). Convective rewarming is traditionally used for rewarming. However, this outside in rewarming makes it difficult to reach sufficient rewarming rates in the sample cores. Although new inside out rewarming technologies such as electromagnetic warming of nanoparticles have recently been developed, they are technically complex and expensive solutions (42). In this regard, the rewarming of vitrified droplets in warm media has the additional advantage in which the rewarmed outer layer is replaced by warm media. This reduces the thermal resistance to the core and the thermal mass of the droplet, significantly increasing the rewarming rate.

The present disclosure provides novel bulk droplet vitrification methods in which we validated the theoretical background and demonstrated the feasibility to use this method to vitrify large cell volumes. Moreover, we showed that this method results in improved hepatocyte viability and metabolic function as compared to conventional cryopreservation. Additional optimization of bulk droplet vitrification can further improve the preservation yield of cells such as human primary hepatocytes. In particular, the pre-incubated CPA concentration could be reduced if the osmotic dehydration prior to vitrification is further optimized, whereby both permeable and non-permeable CPAs should be tested. The method and apparatus described herein can be scaled up to handle large (>1 liter) processing volumes with continuous fluidic low CPA pre-incubation.

### Cryoprotective Agents (CPAs)

Cryoprotectant agents (CPAs) are chemicals, proteins, or polymers that can provide protection to cells from adverse effects of subzero temperatures, e.g., by reducing ice crystal formation within and outside the cells. In some embodiments, a CPA can be dimethyl sulfoxide (DMSO), polyvinylpyrrolidone (PVP), glycerol, ethylene glycol (EG), propylene glycol (PG), propanediol (PROH), methyl pentanediol, polyethylene glycol (PEG), hydroxyethyl starch (HES), sucrose, sucralose, mannitol, maltose, glucose, 3-O-Methyl-D-glucose, trehalose, dextrose, or any combination thereof. One or more different CPAs can be used in the new methods.

In general, the CPAs can include sugars such as trehalose, for the protection of the extracellular compartment and to provide cell membrane stabilization at subzero temperatures. Other sugars include monosaccharides, disaccharides, and trisaccharides such as sucrose, lactulose, lactose, maltose, cellobiose, chitobiose, glucose, galactose, fructose, xylose, mannose, maltose, and raffinose.

In other embodiments, the CPAs include polyethylene glycol (PEG) or other polymers and poloxamers such polypropylene glycol, hydroxyl ethyl starch (HES), gelatin, pluronics, and kolliphor.

The CPAs can also include glycerol or other permeable agents that are freely permeable over plasma membranes such as dimethyl sulfoxide (DMSO), ethylene glycol, propylene glycol, and propanediol.

Alternatively, the CPAs can be or include 3-O-methyl-D-glucose (3-0MG), which accumulates intracellularly, or other non-metabolizable monosaccharides such as Methyl α-D-glucopyranoside, 2,3,4,6-Tetrabenzoyl-D-glucopyranose, Methyl β-D-glucopyranoside, 6-Deoxy-D-glucose, and α-D-Glucopyranose pentabenzoate.

### Systems for Bulk Droplet Vitrification

The systems described herein provide for a vitrification apparatus that can be used to mix cells with a first solution and then a second solution prior to generating droplets, which are dropped into a cooling reservoir containing a cooling liquid, vapor, or gas.

The droplet generation and vitrification apparatus includes a first vessel for containing a first solution, a second vessel for containing a second solution, a mixing and droplet generating chamber, a cooling reservoir containing a cooling liquid, vapor, or gas, and a pressure source. The first and second vessels are connected to the mixing and droplet generating chamber. After the first and second solutions are mixed in the mixing and droplet generating chamber, droplets or cells are expelled through an outlet such that they form droplets. The cell-containing droplets are collected in the cooling reservoir below. A pressure source is arranged such that the cells are incubated in the first solution and mixed with the second solution in less than one minute, e.g., less than 50, 40, 30, 20, 10, or 5 seconds.

As shown in FIG. 1, cells are pre-incubated with a low concentration CPA solution (first cryoprotective solution) and rapidly mixed in a mixing and droplet generating chamber with a high concentration CPA solution (second cryoprotective solution). The mixing dehydrates the cells, which concentrates the pre-incubated intracellular CPA concentration. Droplets containing cells are generated by the mixing and droplet generating chamber, which are directly vitrified in liquid nitrogen before the high CPA concentration can diffuse over the cell membranes.

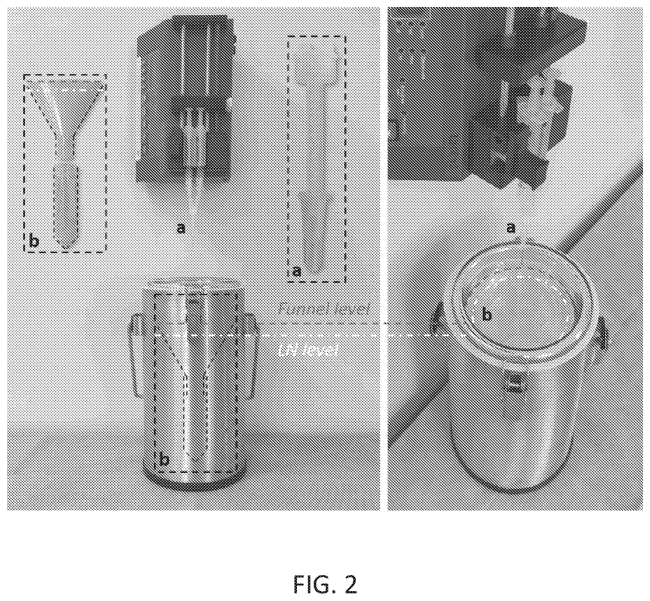

In one embodiment, the droplet generation and vitrification apparatus are arranged such that the first and second vessels are connected to the mixing and droplet generation chamber, which is above the cooling liquid reservoir (FIG. 2). The mixing and droplet generating chamber includes and outlet to expel the droplets. In some embodiments, the cooling liquid reservoir further contains a funnel and container to collect expelled vitrified droplets from the mixing and droplet generation chamber. In one embodiment, the cooling reservoir contains a device to collect specific amounts of bulk droplet volumes.

In one embodiment, the cooling reservoir contains cooling liquid that is agitated to prevent floating or levitation of the droplets on the surface of the liquid, which can be caused by the surface tension effects or the Leidenfrost effect, respectively.

In some embodiments, different cooling mediums are combined to control the cooling rate of the droplets. For example, the droplet is first travels through—and is thereby cooled—the vapor phase of nitrogen and then falls in the liquid phase of nitrogen.

In some embodiments, the droplets are encapsulated in a non-polar liquid to prevent sticking and merging of the droplets during the cooling process.

### EXAMPLES

The invention is further described in the following examples, which do not limit the scope of the invention described in the claims.

The examples disclosed below describe methods of preparing bulk droplet vitrified cells. Bulk droplet vitirification can be used to reduce toxic exposure to CPAs during the cooling process thereby increasing post-preservation viability.

### Example 1: Droplet Size Distribution and Cooling Rate

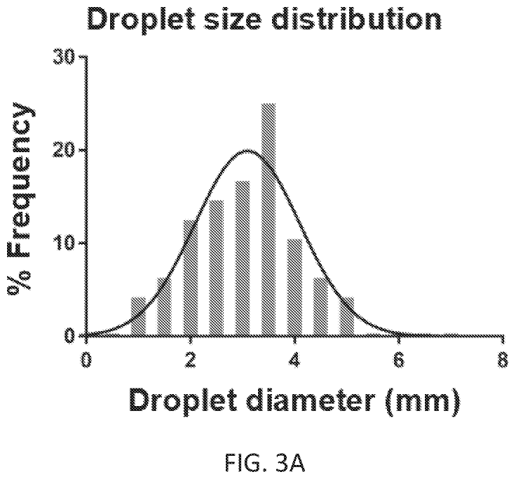

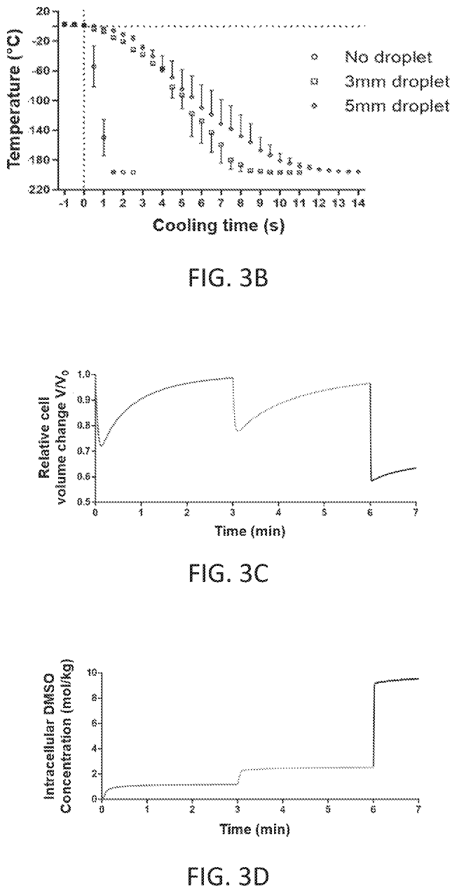

The purpose of Example 1 was to establish average droplet size and cooling rate of droplets generated by the droplet generation and vitrification apparatus described above. We experimentally confirmed droplet sizes and corresponding cooling rates. Droplets without hepatocytes were dropped in liquid nitrogen and collected as described below under “bulk droplet vitrification.” Vitrified droplet diameters were determined by measuring surface area relative to a known surface area (FIG. 3A).

When exposed to a hypertonic solution of permeable CPAs, cells initially lose and then uptake water due to the change in osmotic gradient, accompanied by a constant influx of CPAs. Thus, cell morphology undergoes a shrink-and-swell behavior. The volumetric change as a function of time can be predicted by the K-K formalism (32).

dV/dt=,—LₚART[(mₛ^{e}−mₛⁱ)+σ(m_{c}^{e}−m_{c}ⁱ)] (1)

dn_{c}/dt=(1−σ(½)(m_{c}^{e}+m_{c}ⁱ)dV/dt+PₛA(m_{c}^{e}−m_{c}ⁱ) (2)

In the above formalism, V is the cell volume, A the surface area and n, the content of intracellular CPA. Lₚ is the hydraulic conductivity, P_{S}, the membrane permeability to CPA and σ is the reflection coefficient. R is the gas constant and T the absolute temperature (277 K). m is the molality, with the superscripts denoting intracellular (i) and extracellular (e) and the subscripts denoting non-permeating salt (s) and permeating CPA (c), respectively. The permeating CPAs used during bulk droplet vitrification were DMSO and ethylene glycol (EG), as explained in detail under ‘bulk droplet vitrification’. In our previous study, we have determined L_{P}(1.11 μm/atm/min), P_{S} (7.70 μm/min) and σ (0.581) for rat hepatocytes in exposure to DMSO at 4° C. (19). To simplify our calculation, we substituted DMSO for EG which resulted in a small but acceptable deviance because the relative density difference is less than 1% and (DMSO 1.101 g/cm³ vs EG 1.11 g/cm³) and the size difference of the molecules is very small, assuming a hard sphere model (radius-DMSO 2.9 Angstrom vs radius-EG 2.18-2.44 Angstrom) (33,34). We calculated mₛⁱ=mₛ^{i,0} (V₀−V_{b})/(V−V_{b}) and mᵢ^{c}=n_{c}/(V−V_{b}) where V₀ is the isotonic cell volume, V_{b} the osmotically inactive volume (V_{b}/V₀==0.4061) and mₛ^{i,0} the isotonic salt concentration. The extracellular DMSO concentration in each CPA loading step is 1.175, 2.558 and 9.662 mol/kg, respectively, as approximately converted from the corresponding volume fraction (7.5%, 15% and 40% respectively). Equations (1) and (2) were solved in Matlab™ (The MathWorks, Inc., Natick, Mass.). This simulation was validated in our previous study using real time imaging of rat hepatocytes in a single cell entrapment microfluidic device during exposure to DMSO (19).

To measure the droplet cooling rates, droplets were frozen at the tip of a thin (0.2 mm wire diameter) K-type thermocouple (Omega, Biel, Switzerland). Droplet size was controlled by incrementally freezing additional UW with CPAs on the existing droplet until the desired diameter was obtained. Next, the frozen droplets were rewarmed to 2° C.-4° C. and directly submerged in liquid nitrogen during which the temperature was logged at 100 ms intervals using a USB Thermocouple Data Acquisition Module (Omega, Biel, Switzerland) and Picolog 6 (Picotech, St. Neots, United Kingdom) software. The cooling temperature profiles of 3 mm and 5 mm droplets were measured three times per group (FIG. 3B). Additionally, we measured the cooling rate of the thermocouple without droplets to assess the thermocouple's influence on the cooling rate of the droplets.

As shown in FIG. 3A, the mean droplet diameter was 3.0±1.0 mm (mean±SD). The inverse Leidenfrost effect caused droplets to levitate briefly on the liquid nitrogen surface before submersion. We observed occasional merging of droplets during this phenomenon, resulting in a maximal observed droplet size of 4.4 mm. We measured the cooling rate of 3 mm and 5 mm droplets to determine the mean and minimum cooling rate of the droplets (FIG. 3B). Linear regression gave an average cooling rate of 1320 and 960° C./min respectively.

We simulated the relative volume change of hepatocytes and the intracellular

CPA concentration during CPA pre-incubation and subsequent short exposure to high CPA concentration in the mixing and droplet generation chamber, as shown in FIGS. 3C and 3D. During the mixing time of 1.25 seconds, the dehydration reduces the intracellular volume to 59%. This resulted in an increase of the intracellular CPA concentration from 2.53 to 8.53 Mol/kg H₂O. Additionally, our simulations of cell volume during CPA pre-incubation show that a CPA equilibration period of 3 minutes is enough for hepatocytes to rehydrate to 95% of their initial size.

### Example 2: Post-Preservation Viability and Yield

The purpose of Example 2 was to compare post-preservation viability and yield of cryopreserved and bulk droplet vitrification hepatocytes in suspension and monolayer cultures.

To study the effects of bulk droplet vitrification on primary hepatocyte viability and the potential improvement over cryopreservation, we compared direct post-preservation parameters after bulk droplet vitrification with the most commonly used cryopreservation protocol (30,31). With this cryopreservation protocol, reductions in absolute viability between 15 to 25% have been reported, which is comparable to our observed 22% loss of viability after cryopreservation in this study.

### Methods

Cell Culture Hepatocytes were cultured using a collagen sandwich culture model up to 7 days as described in detail elsewhere (36,37). In short, the hepatocytes were seeded on 12 well precoated collagen plates (Thermo Fischer Scientific) with a seeding density of 1×10⁶ and 9×10⁵ hepatocytes per well for the experimental and fresh control groups respectively, to account for cell death after preservation. Nonattached hepatocytes were washed off one hour after seeding. Collagen top gel was added 24 hours after seeding. Media was changed in 24-hour intervals with a media volume of 0.5 ml per well. Aspirated media was stored at −80° C. for post hoc analysis of urea production and albumin synthesis. Hepatocytes from experimental groups were cultured on the same plates to ensure equal culture conditions.

Cryopreservation

We used the most widely accepted protocol for cryopreservation in literature. Fresh hepatocytes were spun down at 25 g for 5 minutes and resuspended in UW (Bridge to Life, Columbia, South-Carolina) supplemented with 2.2 mg/ml bovine serum albumin (Sigma-Aldrich, Boston, Mass.) and 333 mM glucose (Sigma-Aldrich). DMSO

(Sigma-Aldrich) (5% v/v) was added in two steps with 3 minutes equilibration in between, resulting in a final cell density of 5×10⁶/m1 and a total pre-incubated CPA concentration of 10% v/v DMSO and 300 mM glucose. The hepatocyte suspension was transferred into four 1.5 ml cryovials (ColeParmer, Vernon Hills, Ill.) each containing 7.5×106 hepatocytes. Exactly 3 minutes after the last DMSO addition the vials were placed in a Cryomed™ controlled rate freezer (Thermo Fischer Scientific, Waltham, Mass.) and frozen to −140° C. using the controlled rate freezing protocol as described elsewhere (31). Upon completion, the cryovials were stored at −196° C. until thawing.

After storage at −196° C. for 2 to 8 days, the four cryovials were rapidly thawed in an agitated 37° C. water bath. As soon as all ice was melted the content of the cryovials was added to 25 ml ice cold Dulbecco's Modified Eagle Medium (DMEM) (Sigma-Aldrich) supplemented with 300 mM glucose. After 3 minutes equilibration, the glucose concentration was diluted to 150 mM by addition of 25 ml ice cold DMEM. Subsequently, the cells were spun down for 5 minutes at 25 g and resuspended in 4 ml C+H culture medium (Cell Resource Core, Massachusetts General Hospital, Boston Mass.).

Bulk Droplet Vitrification

Fresh hepatocytes were spun down for 5 minutes at 25 g and resuspended in UW supplemented with 2.4 mg/ml BSA at 4° C. DMSO (3.75% v/v) and Ethylene Glycol (EG) (Sigma-Aldrich) (3.75% v/v) were added in two steps with an equilibration of 3 minutes between each step, resulting in a final cell density of 1e⁷/m1 and a combined pre-incubated CPA concentration of 15%. During the last incubation period, the cells were laden in a 3 ml syringe and a second 3 ml syringe was laden with UW supplemented with 2 mg/ml BSA, 32.5% v/v DMSO, 32.5% v/v EG and 800 mM sucrose. The syringes were mounted into a custom 3D-printed syringe pump adapter that ensures even flowrates from both syringes.

Next, a mixing and droplet generation chamber (Grainger, Lake Forest, Illinois) (FIG. 2) was attached and the complete assembly was placed in the syringe pump (Pumpsystems Inc., Kernersville, N.C.). The syringe pump was mounted in vertical position to the wall of a tissue culture hood with the outlet of the mixing and droplet generation chamber facing down toward the liquid nitrogen dewar (Thermo Fisher Scientific), as shown in FIG. 2. A droplet collection assembly consisting of a funnel (Cole-Parmer) and 50 ml conical tube was placed in the liquid nitrogen dewar as shown in FIG. 2.

At the end of an incubation period of 3 minutes, the syringe pump was started at 2 ml/min, resulting in a high CPA exposure time of 1.25 seconds and a total droplet CPA concentration of 20% v/v DMSO, 20% v/v EG and 400 mM sucrose. The mixing time is dependent on the total flow rate and volume of the mixing and droplet generation chamber. Exactly 5 ml, i.e., 2.5×10⁷ hepatocytes, were dropped in the liquid nitrogen and collected in the conical tube which was stored at −196° C. until rewarming

For rewarming, the cell laden glass droplets were added to 100 ml warm (37° C.) DMEM supplemented with 500 mM sucrose (Sigma-Aldrich) which was agitated for several seconds until the droplets were rewarmed. The resulting cell suspension solution was divided into two 50 ml conical tubes, which were spun down at 50 g for 2 minutes. 37.5 ml was aspirated from both conical tubes, and the sucrose concentration was gradually diluted to 125 mM by the addition of 12.5 ml and 25.0 ml ice cold DMEM every 3 minutes, respectively. Next, the cells were spun down for 5 minutes at 25 g and resuspended and combined in 4 ml C+H culture medium.

Statistical Analysis

Data were tested for normality using visual inspection and the Shapiro-Wilk normality test. Viability data of hepatocytes in suspension before and after preservation were compared using a paired one-way ANOVA with the Tukey correction for multiple testing. The Wilcoxon matched-pairs signed rank test was used to compare the preservation yields and although this data was not normally distributed, we used the F-test to compare the variance in yield. Culture data of fresh cells was corrected to a seeding density of 1 million cells per well to match the experimental groups. The paired Student's t-test was used to compare cell number and viability of monolayer cultured hepatocytes.

### Suspension

Cell membrane integrity is the most widely used metric to assess the quality of preserved cell suspensions. However, the percentage of cells with intact membranes can be paradoxically high if dying cells not only lose their membrane integrity but also disintegrate during preservation. Since this would result in a lower yield, it is important to also consider the preservation yield as an additional preservation parameter.

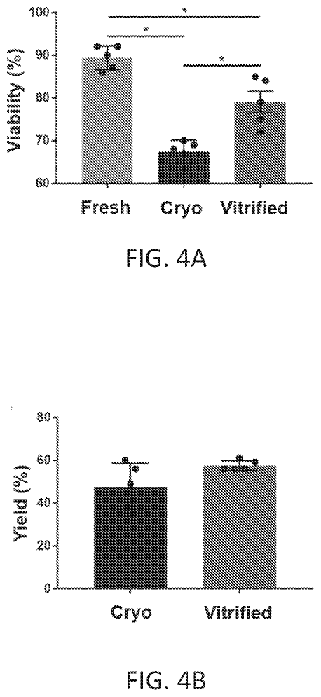

Droplet vitrification resulted in significantly higher membrane integrity of hepatocytes in suspension than cryopreservation (79.0%±2.7% vs 67.4%±5.6%, p=0.044), as shown in FIG. 4A. Moreover, the loss of membrane integrity compared to fresh controls was over two times larger during cryopreservation than during vitrification (mean differences with fresh controls of 22.0%, p=0.002 and 10.4%, p=0.0450 respectively). We did not observe a significant difference in preservation yield between cryopreservation and vitrification (47.6%±11.0% vs 57.64%±2.3%), as shown in FIG. 4B. However, the yield of droplet vitrification was markedly more consistent as compared to cryopreservation, demonstrated by a significantly lower standard deviation (2.3% vs 11.0%, p=0.0108).

When comparing cryopreservation versus our new bulk droplet vitrification approach, loss of hepatocyte viability assessed by membrane integrity testing was about half after droplet vitrification as compared to cryopreservation (10% vs 22%). Yield, defined as the ratio between the total number of live cells after and before preservation, is another important parameter of preservation efficiency, which is often not reported in literature. We observed a 10% higher yield after droplet vitrification, although this did not reach statistical significance. However, it should be noted that there was a significantly more consistent yield after droplet vitrification, which may be important for clinical applications.

### Monolayer

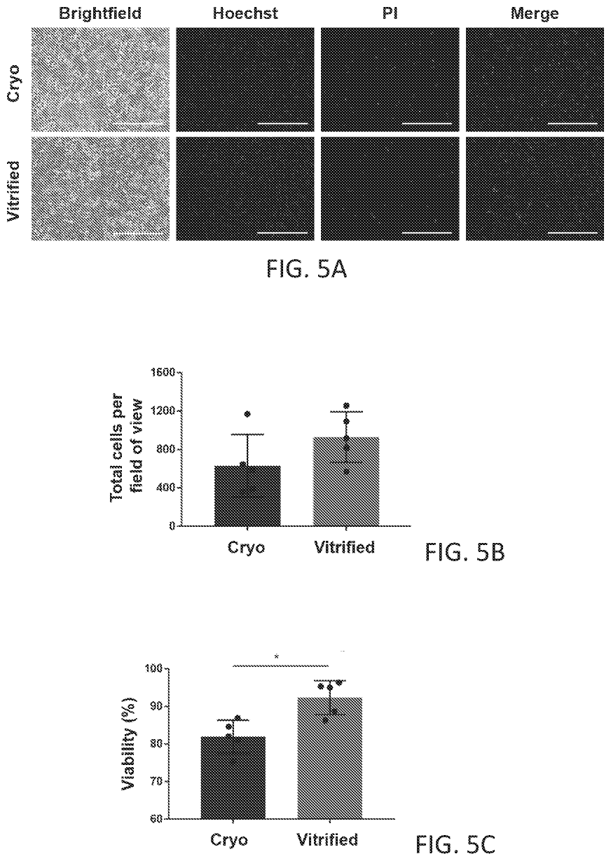

Although direct post-preservation cell membrane integrity is the most widely used metric for cell quality, it results in a potential overestimation of viability because some cells may experience delayed onset cell death. These cells have often lost functional properties such as attachment ability and commonly die within several hours after preservation. We addressed this potential problem by assessing the plating efficiency of cryopreserved and droplet vitrified hepatocytes. We evaluated plating efficiency by dead/live staining of hepatocytes in monoculture one day after preservation (FIG. 5A). Although the difference between the total number of attached cryopreserved and droplet vitrified hepatocytes did not reach statistical significance (930±263 vs 630±327) (FIG. 5B), the mean number of attached hepatocytes was 48% higher after droplet vitrification (FIG. 5C). More importantly, the membrane integrity of the attached hepatocytes after vitrification was significantly higher than after cryopreservation (92.3%±4.5% vs 81.92%±4.4%; p=0.022).

It is well acknowledged that the attachment ability of hepatocytes is reduced after cryopreservation, resulting in lower plating efficiency (46). Therefore, we compared the plating efficiency of hepatocytes in a monolayer 24 hours after seeding. The attachment after droplet vitrification was nearly 50% higher than after cryopreservation. More importantly, the viability of the hepatocytes that did attach was 10% higher after vitrification. This difference is especially important when considering clinical applications; the lower delayed onset cell death after bulk droplet vitrification might result in a more effective therapy with BAL devices and hepatocyte transplantation, with fewer side effects due to remnants of dead hepatocytes.

### Example 3: Viability During Long-Term Collagen Sandwich Cultures

The purpose of Example 3 was to compare long-term collagen sandwich cultures of fresh, cryopreserved, bulk droplet vitrified hepatocytes.

For most applications, high long-term viability and metabolic activity are of utmost importance. To study the long-term effects of preservation on hepatocyte viability and metabolic activity we cultured fresh, cryopreserved and droplet vitrified hepatocytes. The gold standard for primary hepatocyte culture is the collagen sandwich culture whereby the hepatocytes are seeded on collagen coated plates and covered by a collagen top gel 24 hours after seeding (38). We cultured cells using the collagen sandwich model for a week and performed assays from day 2 onwards to allow the hepatocytes to adjust to the collagen top layer. Of note, we did not encounter any contamination issues after direct exposure of hepatocyte droplets to liquid nitrogen, which would have been revealed during a long-term culture.

Statistical analysis methods for visual inspection, viability, preservation yields and culture data were performed as stated above. Time-course data of the Presto Blue assays were analyzed with paired repeated measures two-way ANOVAs with the Tukey correction for multiple testing. P-values <0.05 were considered statistically significant. All analyses were performed in Prism 7.03 (GraphPad Software Inc., La Jolla, California).

Hepatocyte Morphology

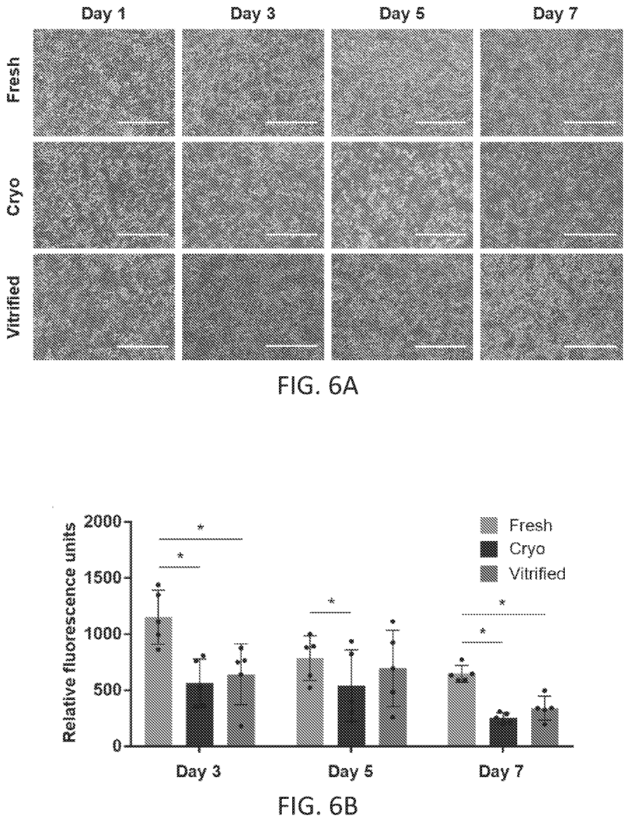

Fresh, cryopreserved and droplet vitrified hepatocytes developed a characteristic polygonal shaped monolayer with the presence of typical binuclear cells on day 1 after plating, as shown in FIG. 6A. The confluency of the cryopreserved hepatocytes was markedly lower compared to the fresh and vitrified hepatocytes, which had similar confluency. On day 3, the hepatocytes of all groups had formed bile cuniculi, which is an important characteristic in collagen sandwich cultures. However, bile cuniculi in the cryopreserved group were less noticeable, as compared to fresh and droplet vitrified cultures, also when lower confluency was considered. Over the course of days, the visible number of dead cells increased in all groups, resulting in a slight reduction in confluency of the fresh and droplet vitrified hepatocytes and an evidently more reduced confluency of the cryopreserved hepatocytes.

Based on phase-contrast images, the droplet vitrified hepatocytes had comparable morphology to fresh plated hepatocytes in long-term collagen sandwich cultures. It should be noted that the fresh hepatocytes were seeded at a lower density than both droplet vitrified and cryopreserved hepatocytes to correct for cell death after preservation. More importantly, the droplet vitrified hepatocytes clearly showed better morphology and confluency than cryopreserved hepatocytes.

### Example 4: Hepatic Function

The purpose of Example 4 was to compare hepatic function during long-term collagen sandwich cultures of fresh, cryopreserved, and bulk droplet vitrified hepatocytes.

### Methods: Assessment of Metabolic Hepatocyte Activity

Reductive Activity

Reductive activity of hepatocytes in collagen sandwich cultures was measured using the Presto Blue essay (Thermo Fisher Scientific). On day 3, 5 and 7, 50 μl Presto blue was added to the media of designated wells. After a 30 minutes incubation, the fluorescence of 110 μL samples was measured on a Synergy-2™ micro plate reader (BioTek, Winooski, Vt.) according to the manufacturer's instructions.

Urea Production

To measure the urea concentration in the culture media, a colorimetric BUN assay was performed with the use of the Stanbio™ BUN Diagnostic Set (Stanbio Laboratories, Cardiff, Wales) with the protocol provided by the manufacturer. Briefly, the urea assay reagent was prepared by mixing one part of the color reagent with two parts of the acid reagent. Standards were prepared and 10 μL of the standards or media samples were plated on a 96 well flat bottom plate, after which 150 μL of the urea reagent mixture was added. After an incubation at 60° C. for 90 minutes, the plate was allowed to cool down (5-10 min) and the absorbance was measured at 520 nm using a Benchmark Plus™ microplate spectrophotometer (Bio-Rad, Hercules, Calif.)

Albumin Synthesis

We measured rat serum albumin in the culture media using an enzyme-linked immunosorbent assay (ELISA) developed in-house. Briefly, high-binding 96 well ELISA plates were coated with rat albumin in PBS and incubated overnight at 4° C. These plates were then washed four times with a 0.5% PBS-Tween solution. 50 μL of standards or media was added to the wells. After diluting 1:10,000 in PBS, peroxidase-conjugated albumin antibody was added to each well and incubated overnight at 4° C./for 2 hours at 37° C. Post incubation, the plates were washed again with 0.5% PBS-Tween. After preparation of o-Phenylenediamine dihydrochloride (400 mg/mL) and 4 uM hydrogen peroxide, 50 μL of the solution was added to each well and incubated for 5 minutes. The reaction was stopped with the addition of 50 μL of 8N sulfuric acid and the absorbance was measured at 490 nm to 650 nm on a Benchmark Plus™ microplate spectrophotometer.

Time-course data of the urea and albumin assays were analyzed with paired repeated measures two-way ANOVAs with the Tukey correction for multiple testing. β-values <0.05 were considered statistically significant. All analyses were performed in Prism 7.03 (GraphPad Software Inc., La Jolla, Calif.).

### Presto Blue Assay

We used the Presto Blue assay to evaluate the general cellular metabolic activity of hepatocytes in collagen sandwich cultures on day 3, 5 and 7 (FIG. 6B and Table 1). Reductive metabolic activity, measured in relative fluorescent units (RFU) of metabolized presto blue per 1×10⁶ hepatocytes, of fresh hepatocytes was significantly higher on all measured days compared to cryopreserved hepatocytes and only on day 3 and 7 compared to vitrified hepatocytes. We did not observe a statistically significant difference between the reductive metabolic activity of droplet vitrified and cryopreserved hepatocytes.

### Urea Production

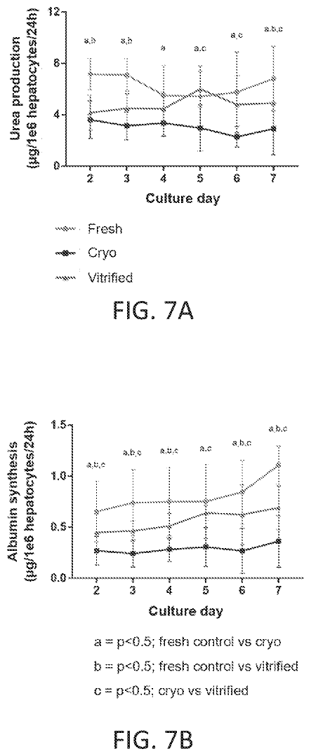

Detoxification is a vital function of the liver. Ammonia is an extremely toxic base which is produced during the deamination of amino acids. Hepatocytes almost exclusively metabolize ammonia into much less toxic urea. As such, urea production is one of the most common markers of specific hepatic function. We measured urea production of fresh, cryopreserved and droplet vitrified hepatocytes in collagen sandwich cultures by sampling the culture media over time for up to one week after preservation (FIG. 7A and Table 1). From day 5 onwards, the urea production of vitrified hepatocytes was significantly higher than the urea production of cryopreserved hepatocytes. Fresh hepatocytes produced significantly more urea on every day compared to cryopreserved hepatocytes. However, in comparison to vitrified hepatocytes, the fresh hepatocytes only produces more urea on day 2, 3 and 7.

### Albumin Synthesis

Albumin is the most abundant blood protein, and is produced almost exclusively by the liver. Therefore, it is considered the most important marker for synthetic metabolism of hepatocytes. We measured the albumin synthesis of fresh, cryopreserved and droplet vitrified hepatocytes in collagen sandwich cultures by sampling the culture media over time up to one week after preservation (FIG. 7B and Table 1). On each day, the albumin synthesis of droplet vitrified hepatocytes was significantly higher than that of cryopreserved hepatocytes. On all days, fresh hepatocytes produced significantly more albumin than both cryopreserved and vitrified hepatocytes, except on day 5.

TABLE 1
Metabolic Activity of Fresh, Cryopreserved, and
Bulk Droplet Vitrified Hepatocytes in Long-Term Collagen Sandwich Cultures

| | | | | | P | | |
|---|---|---|---|---|---|---|---|
| | | | Cryo | | values | | |
| | | Fresh | Mean ± | Vitrified | Fresh- | Fresh- | Cryo- |
| | | Mean ± SD | SD | Mean ± SD | Cryo | Vitrified | Vitrified |
| Presto Blue | Day 3 | 1152 ± 241 | 565 ± 213 | 643 ± 271 | <0.001 | <0.001 | 0.660 |
| Units: RFU | Day 5 | 787 ± 200 | 541 ± 319 | 696 ± 339 | 0.033 | 0.569 | 0.214 |
| | Day 7 | 647 ± 76 | 255 ± 46 | 341 ± 107 | 0.001 | 0.008 | 0.606 |
| Urea | Day 2 | 7.2 ± 1.2 | 3.6 ± 1.5 | 4.2 ± 1.4 | <0.001 | <0.001 | 0.693 |
| production | Day 3 | 7.1 ± 1.3 | 3.1 ± 1.1 | 4.5 ± 1.1 | <0.001 | 0.001 | 0.122 |
| Units: μg/1e6 | Day 4 | 5.5 ± 2.6 | 3.6 ± 1.0 | 4.5 ± 1.3 | 0.007 | 0.271 | 0.232 |
| hepatocytes/24 hr | Day 5 | 5.4 ± 2.4 | 3.0 ± 1.8 | 6.0 ± 1.4 | 0.002 | 0.665 | <0.001 |
| | Day 6 | 5.8 ± 3.1 | 2.3 ± 0.8 | 4.8 ± 2.2 | <0.001 | 0.326 | 0.002 |
| | Day 7 | 6.8 ± 2.5 | 2.9 ± 2.0 | 4.9 ± 1.8 | <0.001 | 0.019 | 0.013 |
| Albumin | Day 2 | 0.65 ± 0.30 | 0.27 ± 0.14 | 0.45 ± 0.18 | <0.001 | 0.019 | 0.045 |
| synthesis | Day 3 | 0.74 ± 0.32 | 0.24 ± 0.13 | 0.46 ± 0.01 | <0.001 | 0.001 | 0.011 |
| Units: μg/1e6 | Day 4 | 0.75 ± 0.33 | 0.28 ± 0.12 | 0.51 ± 0.12 | <0.001 | 0.006 | 0.008 |
| hepatocytes/24 hr | Day 5 | 0.75 ± 0.36 | 0.31 ± 0.19 | 0.64 ± 0.07 | <0.001 | 0.284 | <0.001 |
| | Day 6 | 0.84 ± 0.31 | 0.27 ± 0.22 | 0.62 ± 0.29 | <0.001 | 0.011 | <0.001 |
| | Day 7 | 1.11 ± 0.19 | 0.36 ± 0.25 | 0.69 ± 0.21 | <0.001 | <0.001 | <0.001 |

Note.
Cryo = cryopreserved; Vitrified = bulk droplet vitrified; RFU = relative fluorescence units.

Loss of metabolic activity after cryopreservation is an important problem after cryopreservation of hepatocytes, with negative consequences for clinical applications (4,30,40). In this example, we observed lower metabolic activity after cryopreservation as compared to fresh cultured hepatocytes, demonstrated by both general and liver specific markers of metabolic activity. Bulk droplet vitrified hepatocytes had a significantly higher metabolic activity as compared to cryopreserved hepatocytes, based on a significantly higher urea production and albumin synthesis up to one week after preservation.

### REFERENCES

(1) URL Optn.transplant.hrsa.gov (Accessed 20 November, 2012).
(2) Demetriou, A. A.; Brown, R. S.; Busuttil, R. W.; Fair, J.; McGuire, B. M.; Rosenthal, P.; Am Esch, J. S.; Lerut, J.; Nyberg, S. L.; Salizzoni, M.; et al. Prospective, Randomized, Multicenter, Controlled Trial of a Bioartificial Liver in Treating Acute Liver Failure. Ann. Surg. 2004, 239 (5), 660-667; discussion 667-670.
(3) Sorodoc, L.; Lionte, C.; Sorodoc, V.; Petris, 0.; Jaba, I. Is MARS System Enough for A. Phalloides-Induced Liver Failure Treatment? Hum. Exp. Toxicol. 2010, 29 (10), 823-832.
(4) Carpentier, B.; Gautier, A.; Legallais, C. Artificial and Bioartificial Liver Devices: Present and Future. Gut 2009, 58 (12), 1690-1702.
(5) Fitzpatrick, E.; Mitry, R. R.; Dhawan, A. Human Hepatocyte Transplantation: State of the Art. J. Intern. Med. 2009, 266 (4), 339-357.
(6) Uygun, B. E.; Soto-Gutierrez, A.; Yagi, H.; Izamis, M.-L.; Guzzardi, M. A.; Shulman, C.; Milwid, J.; Kobayashi, N.; Tilles, A.; Berthiaume, F.; et al. Organ Reengineering through Development of a Transplantable Recellularized Liver Graft Using Decellularized Liver Matrix. Nat. Med. 2010, 16 (7), 814-820.
(7) Petersen, T. H.; Calle, E. A.; Zhao, L.; Lee, E. J.; Gui, L.; Raredon, M. B.; Gavrilov, K.; Yi, T.; Zhuang, Z. W.; Breuer, C.; et al. Tissue-Engineered Lungs for in Vivo Implantation. Science 2010, 329 (5991), 538-541.
(8) Ott, H. C.; Matthiesen, T. S.; Goh, S.-K.; Black, L. D.; Kren, S. M.; Netoff, T. I.; Taylor, D. A. Perfusion-Decellularized Matrix: Using Nature's Platform to Engineer a Bioartificial Heart. Nat. Med. 2008, 14 (2), 213-221.
(9) Haridass, D.; Narain, N.; Ott, M. Hepatocyte Transplantation: Waiting for Stem Cells: Curr. Opin. Organ Transplant. 2008, 13 (6), 627-632.
(10) Hughes, R. D.; Mitry, R. R.; Dhawan, A. Current Status of Hepatocyte Transplantation: Transplantation 2012, 93 (4), 342-347.
(11) Cell Injury: Mechanisms, Responses, and Repair; [Result of a Seminar Series Entitled Cell Injury: Responses and Repair Held between March 31st and Juni 2nd, 2004 at the University of Chicago, Chicago Ill.]; Lee, R. C., Seminar Series Entitled Cell Injury: Responses and Repair, Eds.; Annals of the New York Academy of Sciences; New York Academy of Sciences: New York, N.Y, 2005.
(12) Pegg, D. E. Principles of Cryopreservation. Methods Mol. Biol. Clifton N.J. 2007, 368, 39-57.
(13) Baust, J. G.; Gao, D.; Baust, J. M. Cryopreservation: An Emerging Paradigm Change. Organogenesis 2009, 5 (3), 90-96.
(14) Fuller, B.; Morris, G.; Nutt, L.; Attenburrow, V. Functional Recovery of Isolated Rat Hepatocytes upon Thawing from −196 Degrees C. Cryo-Lett. 1980, 1, 139-146.
(15) Fox, I. J.; Chowdhury, J. R. Hepatocyte Transplantation: Hepatocyte Transplantation. Am. J. Transplant. 2004, 4, 7-13.
(16) Fahy, G. M.; MacFarlane, D. R.; Angell, C. A.; Meryman, H. T. Vitrification as an Approach to Cryopreservation. Cryobiology 1984, 21 (4), 407-426.
(17) Hopkins, J. B.; Badeau, R.; Warkentin, M.; Thorne, R. E. Effect of Common Cryoprotectants on Critical Warming Rates and Ice Formation in Aqueous Solutions. Cryobiology 2012, 65 (3), 169-178.
(18) Fahy, G. M.; Wowk, B.; Wu, J.; Phan, J.; Rasch, C.; Chang, A.; Zendejas, E. Cryopreservation of Organs by Vitrification: Perspectives and Recent Advances. Cryobiology 2004, 48 (2), 157-178.
(19) Weng, L.; Ellett, F.; Edd, J.; Wong, K. H. K.; Uygun, K.; Irimia, D.; Stott, S. L.; Toner, M. A Highly-Occupied, Single-Cell Trapping Microarray for Determination of Cell Membrane Permeability. Lab. Chip 2017, 17 (23), 4077-4088.
(20) Pegg, D. E. Principles of Cryopreservation. Methods Mol. Biol. Clifton N.J. 2007, 368, 39-57.
(21) Best, B. P. Cryoprotectant Toxicity: Facts, Issues, and Questions. Rejuvenation Res. 2015, 18 (5), 422-436.
(22) Shi, M.; Ling, K.; Yong, K. W.; Li, Y.; Feng, S.; Zhang, X.; Pingguan-Murphy, B.; Lu, T. J.; Xu, F. High-Throughput Non-Contact Vitrification of Cell-Laden Droplets Based on Cell Printing. Sci. Rep. 2016, 5 (1).
(23) He, X.; Park, E. Y. H.; Fowler, A.; Yarmush, M. L.; Toner, M. Vitrification by Ultra-Fast Cooling at a Low Concentration of Cryoprotectants in a Quartz Micro-Capillary: A Study Using Murine Embryonic Stem Cells. Cryobiology 2008, 56 (3), 223-232.
(24) Kuwayama, M. Highly Efficient Vitrification for Cryopreservation of Human Oocytes and Embryos: The Cryotop Method. Theriogenology 2007, 67 (1), 73-80.
(25) Heo, Y. S.; Nagrath, S.; Moore, A. L.; Zeinali, M.; Irimia, D.; Stott, S. L.; Toth, T. L.; Toner, M. “Universal” vitrification of Cells by Ultra-Fast Cooling. TECHNOLOGY 2015, 03 (01), 64-71.
(26) Demirci, U.; Montesano, G. Cell Encapsulating Droplet Vitrification. Lab. Chip 2007, 7 (11), 1428.
(27) El Assal, R.; Guven, S.; Gurkan, U. A.; Gozen, I.; Shafiee, H.; Dalbeyler, S.; Abdalla, N.; Thomas, G.; Fuld, W.; Illigens, B. M. W.; et al. Bio-Inspired Cryo-Ink Preserves Red Blood Cell Phenotype and Function During Nanoliter Vitrification. Adv. Mater. 2014, 26 (33), 5815-5822.
(28) Dou, R.; Saunders, R. E.; Mohamet, L.; Ward, C. M.; Derby, B. High Throughput Cryopreservation of Cells by Rapid Freezing of Sub-MI Drops Using Inkjet Printing—Cryoprinting. Lab. Chip 2015, 15 (17), 3503-3513.
(29) Samot, J.; Moon, S.; Shao, L.; Zhang, X.; Xu, F.; Song, Y.; Keles, H. 0.; Matloff, L.; Markel, J.; Demirci, U. Blood Banking in Living Droplets. PloS One 2011, 6 (3), e17530.
(30) Stephenne, X.; Najimi, M.; Sokal, E. M. Hepatocyte Cryopreservation: Is It Time to Change the Strategy? World J. Gastroenterol. 2010, 16 (1), 1-14.
(31) Terry, C.; Dhawan, A.; Mitry, R. R.; Lehec, S. C.; Hughes, R. D. Optimization of the Cryopreservation and Thawing Protocol for Human Hepatocytes for Use in Cell Transplantation. Liver Transpl. 2010, 16 (2), 229-237.
(32) Kedem, 0.; Katchalsky, A. Thermodynamic Analysis of the Permeability of Biological Membranes to Non-Electrolytes. Biochim. Biophys. Acta 1958, 27 (2), 229-246.
(33) Schrader, A. M.; Donaldson, S. H.; Song, J.; Cheng, C.-Y.; Lee, D. W.; Han, S.; Israelachvili, J. N. Correlating Steric Hydration Forces with Water Dynamics through Surface Force and Diffusion NMR Measurements in a Lipid-DMSO-H₂O System. Proc. Natl. Acad. Sci. U.S.A 2015, 112 (34), 10708-10713.
(34) Tang, K. E. S.; Bloomfield, V. A. Excluded Volume in Solvation: Sensitivity of Scaled-Particle Theory to Solvent Size and Density. Biophys. J. 2000, 79 (5), 2222-2234.
(35) Seglen, P. 0. Preparation of Isolated Rat Liver Cells. Methods Cell Biol. 1976, 13, 29-83.
(36) Dunn, J. C.; Yarmush, M. L.; Koebe, H. G.; Tompkins, R. G. Hepatocyte Function and Extracellular Matrix Geometry: Long-Term Culture in a Sandwich Configuration. FASEB J Off. Publ. Fed. Am. Soc. Exp. Biol. 1989, 3 (2), 174-177.
(37) Sosef, M. N.; Baust, J. M.; Sugimachi, K.; Fowler, A.; Tompkins, R. G.; Toner, M. Cryopreservation of Isolated Primary Rat Hepatocytes: Enhanced Survival and Long-Term Hepatospecific Function. Ann. Surg. 2005, 241 (1), 125-133.
(38) Dunn, J. C.; Tompkins, R. G.; Yarmush, M. L. Long-Term in Vitro Function of Adult Hepatocytes in a Collagen Sandwich Configuration. Biotechnol. Prog. 1991, 7 (3), 237-245.
(39) Sakiyama, R.; Blau, B. J.; Miki, T. Clinical Translation of Bioartificial Liver Support Systems with Human Pluripotent Stem Cell-Derived Hepatic Cells. World J. Gastroenterol. 2017, 23 (11), 1974.
(40) Pless, G. Artificial and Bioartificial Liver Support. Organogenesis 2007, 3 (1), 20-24.
(41) Parmegiani, L.; Accorsi, A.; Cognigni, G. E.; Bernardi, S.; Troilo, E.; Filicori, M. Sterilization of Liquid Nitrogen with Ultraviolet Irradiation for Safe Vitrification of Human Oocytes or Embryos. Fertil. Steril. 2010, 94 (4), 1525-1528.
(42) Manuchehrabadi, N.; Gao, Z.; Zhang, J.; Ring, H. L.; Shao, Q.; Liu, F.; McDermott, M.; Fok, A.; Rabin, Y.; Brockbank, K. G. M.; et al. Improved Tissue Cryopreservation Using Inductive Heating of Magnetic Nanoparticles. Sci. Transl. Med. 2017, 9 (379).
(43) Wang, X.; Magalhaes, R.; Wu, Y.; Wen, F.; Gouk, S. S.; Watson, P. F.; Yu, H.; Kuleshova, L. L. Development of a Modified Vitrification Strategy Suitable for Subsequent Scale-up for Hepatocyte Preservation. Cryobiology 2012, 65 (3), 289-300.
(44) Jitraruch, S.; Dhawan, A.; Hughes, R. D.; Filippi, C.; Lehec, S. C.; Glover, L.; Mitry, R. R. Cryopreservation of Hepatocyte Microbeads for Clinical Transplantation. Cell Transplant. 2017, 26 (8), 1341-1354.
(45) Arikura, J.; Kobayashi, N.; Okitsu, T.; Noguchi, H.; Totsugawa, T.; Watanabe, T.; Matsumura, T.; Maruyama, M.; Kosaka, Y.; Tanaka, N.; et al. U W Solution: A Promising Tool for Cryopreservation of Primarily Isolated Rat Hepatocytes. J. Hepatobiliary. Pancreat. Surg. 2002, 9 (6), 742-749.
(46) Guillouzo, A.; Rialland, L.; Fautrel, A.; Guyomard, C. Survival and Function of Isolated Hepatocytes after Cryopreservation. Chem. Biol. Interact. 1999, 121 (1), 7-16.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of bulk droplet vitrification of cells, the method comprising:
(a) incubating a plurality of cells in a first cryoprotective solution comprising one or more cryoprotectant agents (CPAs) at a concentration of about 20% or less (v/v);
(b) mixing the plurality of cells in the first cryoprotective solution with a second cryoprotective solution comprising one or more CPAs at a concentration of greater than about 30% (v/v) and generating a plurality of droplets of the resulting mixture within less than one minute from the start of mixing, wherein at least some of the droplets contain one or more of the cells; and
(c) rapidly cooling the plurality of droplets in a cooling liquid at a cooling rate of faster than 0.1° C./second for a time sufficient to bulk vitrify the droplets comprising cells.

2. The method of claim 1, wherein the concentration of the CPA of the first solution is less than about 15% (v/v).

3. The method of claim 1, wherein droplets in the plurality of droplets have an average diameter of between about 0.5 mm and about 10 mm.

4. The method of claim 1, wherein the cells comprise hepatocytes.

5. The method of claim 1, wherein the first solution comprises between 5% (v/v) and 10% (v/v) dimethyl sulfoxide (DMSO) and between 5% (v/v) and 10% (v/v) ethylene glycol.

6. The method of claim 1, wherein the first solution comprises about 7.5% (v/v) DMSO and about 7.5% (v/v) ethylene glycol.

7. The method of any one of claim 1, wherein the second solution comprises greater than 20% (v/v) DMSO, greater than 20% (v/v) ethylene glycol, and greater than 500 mM sucrose.

8. (canceled)

9. The method of claim 1, where the first and/or second solution further comprises University of Wisconsin solution (UW solution) and/or bovine serum albumin (BSA).

10. (canceled)

11. The method of any one of claim 1, wherein the mixing and droplet formation occurs in less than 5 seconds.

12. The method of claim 1, wherein the droplets comprise between 10-30% (v/v) DMSO, about 10-30% (v/v) ethylene glycol, and about 200-600 mM sucrose.

13. (canceled)

14. The method of claim 1, wherein the cooling rate is between about 900° C./min and 1400° C./min.

15. The method of claim 1, wherein the droplets are cooled to a temperature of about −180° C. to about −210° C.

16. The method of claim 1, wherein the cooling liquid comprises liquid nitrogen.

17. The method of claim 1, wherein the vitrified cells have greater than 75% cell viability after rewarming, as measured by assessing membrane integrity of the cells.

18. The method of claim 1, wherein the vitrified droplets are generated continuously from the mixture of the first solution and the second solution at a volumetric flow rate of least 4 ml/minute of the mixture being used to form the vitrified droplets per minute.

19. A droplet generation and vitrification system comprising
a first vessel for containing a first solution;
a second vessel for containing a second solution;
a mixing and droplet generation chamber comprising an inlet connected to both the first vessel and the second vessel and further comprising an outlet, wherein the mixing and droplet generation chamber is configured to receive and mix the first solution and the second solution and to expel through the outlet droplets of the mixture of the first solution and the second solution;
a cooling liquid reservoir arranged to receive droplets expelled from the mixing and droplet generating chamber outlet; and
a pressure source arranged to flow the first and second solutions from the first and second vessels into and through the mixing and droplet generation chamber, and controlled to flow the mixture from the inlet to the outlet of the mixing and droplet generation chamber within less than 10 seconds.

20. The system of claim 19, wherein the first and second vessels and the mixing and droplet generation chamber outlet are arranged a distance above the cooling container, such that the droplets fall from the outlet of the mixing and droplet generation chamber into the cooling liquid reservoir.

21. The system of claim 19, wherein the outlet of the mixing and droplet generation chamber is sized, and the pressure source is controlled, to generate droplets with an average diameter of between about 0.5 mm and about 10 mm.

22. The system of claim 19, wherein the pressure source is controlled to flow the mixture from the inlet to the outlet of the mixing and droplet generation chamber within less than 2 seconds.

23. The system of claim 19, wherein the cooling liquid reservoir contains one or more of liquid nitrogen, liquid isopentane, and liquid propane at a temperature of between about −180° C. and about −210° C.

24. A composition comprising a plurality of vitrified droplets made by the method of claim 1.

25. The composition of claim 24, wherein the droplets have an average diameter of between about 0.5 mm and about 10 mm.

26. The composition of claim 24, wherein the droplets comprise hepatocytes.

27. The composition of claim 24, wherein the droplets comprise between 10-30% (v/v) DMSO, about 10-30% (v/v) ethylene glycol, and about 200-600 mM sucrose.

28. (canceled)

29. The composition of claim 24, wherein the vitrified cells have greater than 75% cell viability, as measured by assessing membrane integrity of the cells.
